(19) United States
(12) Patent Application Publication
(10) Pub. No.: US 2007/0027205 A1
(43) Pub. Date: Feb. 1, 2007

(54) Diarylmethyl derivatives as potassium channel modulators

(76) Inventors: Joachim Demnitz, Kobenhayn (DK); Dorte Stobaek, Farum (DK); Lars Siim Madsen, Smorum (DK); Soren Peter Olesen, Klampenborg (DK)

Correspondence Address: BIRCH STEWART KOLASCH & BIRCH, PO BOX 747, FALLS CHURCH, VA, 22040-0747, US

(21) Appl. No.: 10/561,436

(22) PCT Filed: Jun. 30, 2004
(86) PCT No.: PCT/EP04/51303

§ 371 Date: Dec. 20, 2005

(30) Foreign Application Priority Data
Jul. 3, 2003 (DK) PA2003 01016

(51) Int. Cl. A61K 31/4015; A61K 31/277

(52) U.S. Cl. 514/425; 514/521; 558/410; 548/546

(57) ABSTRACT

This invention relates to novel compounds useful as potassium channel modulators. More specifically the invention provides chemical compounds useful as modulators of SK_{Ca} and/or IK_{Ca} channels.

### TECHNICAL FIELD

This invention relates to novel compounds useful as potassium channel modulators. More specifically the invention provides chemical compounds useful as modulators of SK_{Ca} and/or IK_{Ca} channels.

### BACKGROUND ART

Ion channels are transmembrane proteins, which catalyse the transport of inorganic ions across cell membranes. The ion channels participate in processes as diverse as the generation and timing of action potentials, synaptic transmissions, secretion of hormones, contraction of muscles, etc.

Two types of Ca²⁺-activated potassium channels have been described from lymphocytes: 1) Small-conductance, apamin-sensitive, Ca²⁺-activated K-channels (SK_{Ca}) and 2) Intermediate-conductance, inwardly rectifying, Clotrimazole-sensitive, Ca²⁺-activated K-channels (IK_{Ca}), also referred to as Gardos-channels.

WO 97/34589 describes triaryl methane compounds that inhibit mammalian cell proliferation, inhibit the Gardos channel of erythrocytes, reduce sickle erythrocyte dehydration and/or delay the occurrence of erythrocyte sickling or deformation, and suggest the use of these compounds in abnormal cell proliferation.

WO 97/34599 describes the use of Clotrimazol and related compounds in the treatment of diarrhoea.

WO 00/50026 describes Gardos channel antagonists (i.e. Ca²⁺-activated K-channels), which inhibit the Gardos channel of erythrocytes, reduce sickle erythrocyte dehydration and/or delay the occurrence of erythrocyte sickling or deformation.

WO 01/27070 describes the use of carbonylamino derivatives for treating CNS disorders relating to metabotropic glutamate receptor antagonists and/or agonists.

WO 01/49663 describes the use of certain substituted triarylmethane compounds for immunosuppressive treatment of autoimmune disorders or inflammatory diseases.

### SUMMARY OF THE INVENTION

According to the present invention it has now been found that a particular group of chemical compounds possess valuable activity as modulators of SK_{Ca} and/or IK_{Ca} channels.

Therefore, in its first aspect, the invention provides diaryl methyl derivatives of Formula I

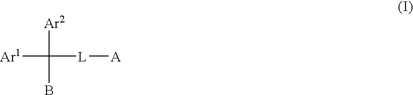

or a pharmaceutically-acceptable addition salt thereof, wherein,

Ar¹ and Ar², independently of one another, represent an aromatic arbocyclic or heterocyclic monocyclic group, which aromatic carbocyclic or heterocyclic monocyclic group is optionally substituted one or more times with substituents selected from the group consisting of alkyl, alkoxy, halo, haloalkyl, haloalkoxy, cyano and nitro;

L is absent (i.e. represents a single bond) or represents a linker selected from the group consisting of —CH₂—, —CH₂CH₂—, —CH(CH₃)—, —CH₂CH₂CH₂—, —CH₂CH(CH₃)—, —S—, —S—CH₂—, —S—CH₂CH₂—, —S—CH(CH₃)—, —S—CH₂CH₂CH₂—, —S—CH₂CH(CH₃)—, —NH—, —NH—CH₂—, —NH—CH₂CH₂—, —NH—CH(CH₃)—, —NH—CH₂CH₂CH₂— and —NH—CH₂CH(CH₃)—; and

A and B, independently of one another, represent —CN; —COOR′, —CONR′R″, —C(═NOR′)R″ or —C(═NOR′)NR″R′″, wherein R′, R″ and R′″, independently of one another, represent hydrogen or alkyl; pyridinyl, phenyl, —SO₂-phenyl or —O—SO₂-phenyl, which phenyl group may optionally be substituted one or more times with substituents selected from the group consisting of alkyl, alkoxy, halo, haloalkyl, haloalkoxy, cyano and nitro; or

A represents —CN; —COOR′, —CONR′R″, —C(═NOR′)R″ or —C(═NOR′)NR″R′″, wherein R′, R″ and R′″, independently of one another, represent hydrogen or alkyl; pyridinyl, —SO₂-phenyl or —O—SO₂-phenyl, which phenyl group may optionally be substituted with alkyl, alkoxy, halo, haloalkyl, haloalkoxy, cyano and nitro; and B represents —CN, —COOR′ or —CONR′R″, wherein R′ and R″, independently of one another, represent hydrogen or alkyl; a phenyl group, which phenyl group is optionally substituted one or two times with alkyl, alkoxy, halo, haloalkyl, haloalkoxy, cyano and/or nitro; or a pyridinyl group, which pyridinyl group is optionally substituted one or two times with alkyl, alkoxy, halo, haloalkyl, haloalkoxy, cyano and/or nitro.

In another aspect the invention provides pharmaceutical compositions comprising a therapeutically effective amount of a chemical compound of the invention, or a pharmaceutically-acceptable addition salt thereof, together with at least one pharmaceutically-acceptable carrier or diluent.

In yet another aspect the invention relates to the use of a chemical compound of the invention, or a pharmaceutically-acceptable addition salt thereof, for the manufacture of a medicament.

In still another aspect the invention provides methods for treatment, prevention or alleviation of diseases or disorders or conditions responsive to modulation of SK_{Ca} and/or IK_{Ca} channels, which method comprises the step of administering to such a living animal body in need thereof a therapeutically effective amount of a compound of the invention or a pharmaceutically-acceptable addition salt thereof.

Other objects of the invention will be apparent to the person skilled in the art from the following detailed description and examples.

### DETAILED DISCLOSURE OF THE INVENTION

The present invention provides novel diaryl methyl derivatives of Formula I

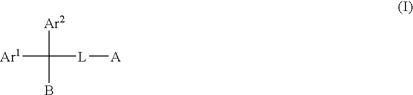

or a pharmaceutically-acceptable addition salt thereof, wherein,

Ar¹ and Ar², independently of one another, represent an aromatic carbocyclic or heterocyclic monocyclic group, which aromatic carbocyclic or heterocyclic monocyclic group is optionally substituted one or more times with substituents selected from the group consisting of alkyl, alkoxy, halo, haloalkyl, haloalkoxy, cyano and nitro;

L is absent (i.e. represents a single bond) or represents a linker selected from the group consisting of —CH₂—, —CH₂CH₂—, —CH(CH₃)—, —CH₂CH₂CH₂—, —CH₂CH(CH₃)—, —S—, —S—CH₂—, —S—CH₂CH₂—, —S—CH(CH₃)—, —S—CH₂CH₂CH₂—, —S—CH₂CH(CH₃)—, —NH—, —NH—CH₂—, —NH—CH₂CH₂—, —NH—CH(CH₃)—, —NH—CH₂CH₂CH₂— and —NH—CH₂CH(CH₃)—; and

A and B, independently of one another, represent —CN; —COOR′, —CONR′R″, —C(═NOR′)R″ or —C(═NOR′)NR″R′″, wherein R′, R″ and R′″, independently of one another, represent hydrogen or alkyl; pyridinyl, phenyl, —SO₂-phenyl or —O—SO₂-phenyl, which phenyl group may optionally be substituted one or more times with substituents selected from the group consisting of alkyl, alkoxy, halo, haloalkyl, haloalkoxy, cyano and nitro; or

A represents —CN; —COOR′, —CONR′R″, —C(═NOR′)R″ or —C(═NOR′)NR″R′″, wherein R′, R″ and R′″, independently of one another, represent hydrogen or alkyl; pyridinyl, —SO₂-phenyl or —O—SO₂-phenyl, which phenyl group may optionally be substituted with alkyl, alkoxy, halo, haloalkyl, haloalkoxy, cyano and nitro; and B represents —CN, —COOR′ or —CONR′R″, wherein R′ and R″, independently of one another, represent hydrogen or alkyl; a phenyl group, which phenyl group is optionally substituted one or two times with alkyl, alkoxy, halo, haloalkyl, haloalkoxy, cyano and/or nitro; or a pyridinyl group, which pyridinyl group is optionally substituted one or two times with alkyl, alkoxy, halo, haloalkyl, haloalkoxy, cyano and/or nitro.

In another preferred embodiment the diaryl methyl derivative of the invention is a compound of Formula I, wherein

Ar¹ and Ar², independently of one another, represent an aromatic carbocyclic or heterocyclic monocyclic group, which aromatic carbocyclic or heterocyclic monocyclic group is optionally substituted one or more times with substituents selected from the group consisting of alkyl, alkoxy, halo, haloalkyl, haloalkoxy, cyano and nitro; and

L is absent (i.e. represents a single bond) or represents a linker selected from the group consisting of —CH₂—, —CH₂CH₂—, —CH(CH₃)—, —CH₂CH₂CH₂—, —CH₂CH(CH₃)—, —S—, —S—CH₂—, —S—CH₂CH₂—, —S—CH(CH₃)—, —S—CH₂CH₂CH₂—, —S—CH₂CH(CH₃)—, —NH—, —NH—CH₂—, —NH—CH₂CH₂—, —NH—CH(CH₃)—, —NH—CH₂CH₂CH₂— and —NH—CH₂CH(CH₃)—; and

A and B, independently of one another, represent —CN, —COOR′, —CONR′R″, —C(═NOR′)R″, —C(═NOR′)NR″R′″, wherein R′, R″ and R′″, independently of one another, represent hydrogen or alkyl, —SO₂-phenyl, —O—SO₂-phenyl, which phenyl group may optionally be substituted one or more times with substituents selected from the group consisting of alkyl, alkoxy, halo, haloalkyl, haloalkoxy, cyano and nitro; or

A represents —CN, —COOR′, —CON R′R″, —C(═NOR′)R″, —C(═NOR′)NR″R′″, wherein R′, R″ and R′″, independently of one another, represent hydrogen or alkyl, —SO₂-phenyl, —O—SO₂-phenyl, which phenyl group may optionally be substituted with alkyl, alkoxy, halo, haloalkyl, haloalkoxy, cyano and nitro; and

B represents an alkyl or phenyl group, which phenyl group is optionally substituted one or two times with alkyl, alkoxy, halo, haloalkyl, haloalkoxy, cyano and/or nitro, a pyridinyl group, which pyridinyl group is optionally substituted one or two times with alkyl, alkoxy, halo, haloalkyl, haloalkoxy, cyano and/or nitro.

In another preferred embodiment the diary methyl derivative of the invention is a compound of Formula I, wherein L represents a linker selected from the group consisting of —CH₂—, —CH₂CH₂—, —CH₂CH₂CH₂—, —CH₂CH(CH₃)—, —S—, —S—CH₂— and —S— CH(CH₃)—.

In a third preferred embodiment the diaryl methyl derivatives of the invention is a compound of Formula I, wherein L is absent (i.e. represents a single bond); or L represents a linker selected from the group consisting of —CH₂—, —CH₂CH₂—, —CH₂CH₂CH₂—, —CH₂CH(CH₃)—, —S—, —S—CH₂— and —S—CH(CH₃)—.

In a fourth preferred embodiment the diaryl methyl derivatives of the invention is a compound of Formula I, wherein A and B, independently of one another, represent —CN; —COOR′, —CONR′R″, —C(═NOR′)R″ or —C(═NOR′)NR″R′″, wherein R′, R″ and R′″, independently of one another, represent hydrogen or alkyl; pyridinyl, phenyl, —SO₂-phenyl or —O—SO₂-phenyl, which phenyl group may optionally be substituted one or more times with substituents selected from the group consisting of alkyl, alkoxy, halo, haloalkyl, haloalkoxy, cyano and nitro; or A represents —CN; —COOR′, —CONR′R″, —C(═NOR′)R″ or —C(═NOR′)NR″R′″, wherein R′, R″ and R′″, independently of one another, represent hydrogen or alkyl; pyridinyl, —SO₂-phenyl or —O—SO₂-phenyl, which phenyl group may optionally be substituted with alkyl, alkoxy, halo, haloalkyl, haloalkoxy, cyano and nitro; and B represents —CN, —COOR′ or —CONR′R″, wherein R′ and R″, independently of one another, represent hydrogen or alkyl; a phenyl group, which phenyl group is optionally substituted one or two times with alkyl, alkoxy, halo, haloalkyl, haloalkoxy, cyano and/or nitro; or a pyridinyl group, which pyridinyl group is optionally substituted one or two times with alkyl, alkoxy, halo, haloalkyl, haloalkoxy, cyano and/or nitro.

In a more preferred embodiment A represents —CN; —COOR′, —CONR′R″, —C(═NOR′)R″ or —C(═NOR′)NR″R′″, wherein R′, R″ and R′″, independently of one another, represent hydrogen or alkyl; pyridinyl, —SO₂-phenyl or —O—SO₂-phenyl, which phenyl group may optionally be substituted with alkyl, alkoxy, halo, haloalkyl, haloalkoxy, cyano and nitro; and B represents —CN, —COOR′ or —CONR′R″, wherein R′ and R″, independently of one another, represent hydrogen or alkyl; a phenyl group, which phenyl group is optionally substituted one or two times with alkyl, alkoxy, halo, haloalkyl, haloalkoxy, cyano and/or nitro; or a pyridinyl group, which pyridinyl group is optionally substituted one or two times with alkyl, alkoxy, halo, haloalkyl, haloalkoxy, cyano and/or nitro.

In an even more preferred embodiment A represents —COOH, —COOCH₃, —COOCH₂CH₃, —CONH₂, —C(═NOH)NH₂, pyridinyl, or —O—SO₂-phenyl, which phenyl group is substituted with alkyl or halo, haloalkyl, cyano or nitro; and B represents —CONH₂, —CN, or a phenyl group substituted with fluoro.

In a still more preferred embodiment A represents —COOH, —COOCH₃, —COOCH₂CH₃, —CONH₂, —C(═NOH)NH₂; and B represents —CONH₂, —CN.

In a yet still more preferred embodiment A represents pyridin-2-yl, pyridin-3-yl, pyridin-4-yl or —O—SO₂-phenyl, which phenyl group is substituted with methyl or ethyl; and B represents —CN.

In a fifth preferred embodiment the diaryl methyl derivatives of the invention is a compound of Formula I, wherein A and B, independently of one another, represent —CN, —COOR′, —CONR′R″, —C(═NOR′)R″, —C(═NOR′)NR″R′″, wherein R′, R″ and R′″, independently of one another, represent hydrogen or alkyl, —SO₂-phenyl, —O—SO₂— phenyl, which phenyl group may optionally be substituted with alkyl and/or alkoxy; or

A represents —CN, —COOR′, —CONR′R″, —C(═NOR′)R″, —C(═NOR′)NR″R′″, wherein R′, R″ and R′″, independently of one another, represent hydrogen or alkyl, —SO₂-phenyl, —O—SO₂-phenyl, which phenyl group may optionally be substituted with alkyl and/or alkoxy; and B represents an alkyl or phenyl group, which phenyl group is optionally substituted one or two times with halo; or a pyridinyl group.

In a sixth preferred embodiment the diaryl methyl derivatives of the invention is a compound of Formula I, wherein Ar¹ and Ar², independently of one another, represent a phenyl group, which phenyl group is substituted one or two times with alkyl, alkoxy, halo, haloalkyl, cyano and/or nitro; or a pyridinyl group, which pyridinyl group is optionally substituted one or two times with alkyl, alkoxy, halo, haloalkyl, cyano and/or nitro.

In a more preferred embodiment both of Ar¹ and Ar² represent a phenyl group, which phenyl groups, independently of one another, are substituted one or two times with halo, haloalkyl, cyano and/or nitro; or both of Ar¹ and Ar² represent a pyridinyl group, which pyridinyl groups, independently of one another, are optionally substituted one or two times with halo, haloalkyl, cyano and/or nitro.

In an even more preferred embodiment both of Ar¹ and Ar² represent a halo-substituted phenyl group.

In a still more preferred embodiment L represents —CH₂—, —CH₂CH₂—, —CH₂CH₂CH₂—, —CH₂CH(CH₃)—, —S—, —S—CH₂— or —S—CH(CH₃)—.

In a still even more preferred embodiment A represents —COOH, —COOCH₃, —COOCH₂CH₃, —CONH₂, —C(═NOH)NH₂; and B represents —CONH₂, —CN.

In a most preferred embodiment the diaryl methyl derivatives of the invention is

2,2-Bis-(4-fluoro-phenyl)-succinamide;

3-Cyano-3,3-bis-(4-fluoro-phenyl)-propionic acid;

p-Toluensulfonic acid 2cyano-2,2-bis-(4-fluoro-phenyl)-ethyl ester;

Ethyl 4-cyano-4,4-bis-(4-fluoro-phenyl) butyrate; or

2-[(2-Fluoro-phenyl)-bis-(4-fluoro-phenyl)-methanesulfanyl]-N-hydroxy acetamidine;

or a pharmaceutically-acceptable addition salt thereof.

In a seventh preferred embodiment the diaryl methyl derivatives of the invention is a compound of Formula I, wherein both of Ar¹ and Ar² represent a phenyl group, which phenyl groups, independently of one another, are substituted one or two times with halo, haloalkyl, cyano and/or nitro.

In a more preferred embodiment L represents —CH₂—, —CH₂CH₂—, —CH₂CH₂CH₂—, —CH₂CH(CH₃)—, —S—, —S—CH₂— or —S—CH(CH₃)—.

In an even more preferred embodiment A represents pyridinyl, in particular pyridin-2-yl, pyridin-3yl or pyridin-4-yl; and B represents —CONH₂, —CN.

In a most preferred embodiment the diaryl methyl derivatives of the invention is

2-(4-Fluoro-phenyl)-2-(4nitro-3-trifluoromethyl-phenyl)-3-pyridin-2-yl-propionitrile;

or a pharmaceutically-acceptable addition salt thereof.

In an eight preferred embodiment the diaryl methyl derivatives of the invention is a compound of Formula I, wherein both of Ar¹ and Ar² represent a pyridinyl, in particular a pyridin-2-yl, a pyridin-3-yl or a pyridin-4-yl group.

In a more preferred embodiment L represents —CH₂—, —CH₂CH₂—, —CH₂CH₂CH₂—, —CH₂CH(CH₃)—, —S—, —S—CH₂— or —S—CH(CH₃)—.

In an even more preferred embodiment A represents —COOH, —COOCH₃, —COOCH₂CH₃, —CONH₂, —C(═NOH)NH₂; and B represents —CONH₂, —CN.

In a most preferred embodiment the diaryl methyl derivatives of the invention is

Methyl 4-cyano-4,4-bis-(pyridin-2-yl) butyrate; or

Methyl 4-cyano-2-methyl-4,4-bis-(pyridin-2-yl) butyrate;

or a pharmaceutically-acceptable addition salt thereof.

In a ninth preferred embodiment the diaryl methyl derivatives of the invention is a compound of Formula I, wherein Ar¹ and Ar², independently of one another, represent a phenyl group, which phenyl group is optionally substituted one or two times with alkyl, alkoxy, halo, haloalkyl, cyano and/or nitro; or a pyridinyl group, which pyridinyl group is optionally substituted one or two times with alkyl, alkoxy, halo, haloalkyl, cyano and/or nitro.

In a tenth preferred embodiment the diaryl methyl derivatives of the invention is a compound of Formula I, wherein both of Ar¹ and Ar² represent a phenyl group, which phenyl groups, independently of one another, are optionally substituted one or two times with halo, haloalkyl, cyano and/or nitro; or both of Ar¹ and Ar² represent a pyridinyl group, which pyridinyl groups, independently of one another, are optionally substituted one or two times with halo, haloalkyl, cyano and/or nitro; or one of Ar¹ and Ar² represents a phenyl group, which phenyl group is optionally substituted one or two times with halo, haloalkyl, cyano and/or nitro; and the other of Ar¹ and Ar² represents a pyridinyl group, which pyridinyl group is optionally substituted one or two times with halo, haloalkyl, cyano and/or nitro.

Any combination of two or more of the embodiments described herein is considered within the scope of the present invention.

Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl and tertiary butyl. In another preferred embodiment of this invention alkyl represents a C₁₋₃-alkyl group, which may in particular be methyl, ethyl, propyl or isopropyl.

In the context of this invention a haloalkyl group designates an alkyl group as defined herein, which alkyl group is substituted one or more times with halogen. Preferred haloalkyl groups of the invention include trihalogenmethyl, preferably CF₃.

In the context of this invention an alkoxy group designates an “alkyl-O—” group, wherein alkyl is as defined above. Examples of preferred alkoxy groups of the invention include methoxy and ethoxy.

In the context of this invention a haloalkoxy group designates an alkoxy group as defined herein, which alkoxy group is substituted one or more times with halo. Preferred haloalkoxy groups of the invention include trihalogenmethoxy, preferably CF₃O—.

In the context of this invention an aromatic carbocyclic group designates a monocyclic or polycyclic aromatic hydrocarbon (aryl) group. Examples of preferred aryl groups of the invention include phenyl, indenyl, naphthyl, azulenyl, fluorenyl and anthracenyl. In a most preferred embodiment an aryl group of the invention is phenyl.

In the context of this invention an aromatic heterocyclic monocyclic group is a heteroaryl, which holds one or more heteroatoms in its ring structure. Preferred heteroatoms include nitrogen (N), oxygen (O) and sulphur (S).

Preferred monocyclic heteroaryl groups of the invention include aromatic 5- and 6 membered heterocyclic monocyclic groups, including furanyl, in particular 2— or 3-furanyl; thienyl, in particular 2 or 3-thienyl; selenophenyl, in particular 2— or 3-selenophenyl; pyrrolyl (azolyl), In particular 2 or 3-pyrrolyl; oxazolyl, in particular oxazol-2,4 or 5-yl; thiazolyl, in particular thiazol-2,4 or 5-yl; imidazolyl, in particular 2 or 4-imidazolyl; pyrazolyl, in particular 1,3 or 4-pyrazolyl; isoxazolyl, in particular isoxazol-3,4 or 5-yl; isothiazolyl, in particular isothiazol-3,4 or 5-yl; oxadiazolyl, in particular 1,2,3-oxadiazol-4 or 5-yl, or 1,3,4-oxadiazol-2-yl; triazolyl, in particular 1,2,3-triazol-4-yl or 1,2,4-triazol-3-yl; thiadiazolyl, in particular 1,2,3-thiadiazol-4 or 5-yl, or 1,3,4-thiadiazol-2-yl; pyridinyl, in particular 2,3 or 4-pyridinyl; pyridazinyl, in particular 3 or 4-pyridazinyl; pyrimidinyl, in particular 2,4 or 5-pyrimidinyl; pyrazinyl, in particular 2 or 3-pyrazinyl; and triazinyl, in particular 1,2,4- or 1,3,5-triazinyl.

Most preferred monocyclic heteroaryl groups of the invention include pyridinyl, in particular 2,3 or 4-pyridinyl.

Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a chemical compound of the invention include alkali metal salts such as the sodium salt of a chemical compound of the invention containing a carboxy group.

Steric Isomers

The chemical compounds of the invention may exist in (+) and (−) forms as well as in racemic forms (±). The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystalilsation of d- or I- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

Biological Activity

According to the present invention it has now been found that the chemical compounds of the invention possess valuable activity as modulators of SK_{Ca} and/or IK_{Ca} channels, in particular by having an inhibitory activity.

The SK/IK/BK channel modulating or inhibiting activity may be monitored using conventional electrophysiological methods such as patch-clamp techniques, or conventional spectroscopic methods such as FLIPR assay (Fluorescence Image Plate Reader; available from Molecular Devices). These methods generally comprises subjecting an SK_{Ca} and/or IK_{Ca} containing cell to the action of the chemical compound of the invention, followed by monitoring the membrane potential of the SK_{Ca} and/or IK_{Ca} containing cell in order to identify changes in the membrane potential caused by the action of the compound of the invention. Using such methods the chemical compounds of the invention show IK_{Ca} inhibitory activity in concentrations below 100 μM, preferably below 10 μM, more preferred below 1 μm. In its most preferred embodiment compounds show IK_{Ca} inhibitory activity show activity in low micromolar and the nanomolar range.

Based on their biological activity the compounds of the invention are considered useful for the for the treatment, prevention or alleviation of a disease or a disorder or a condition of a mammal, including a human, which disease, disorder or condition is responsive to modulation of SK_{Ca} and/or IK_{Ca} channels, including diseases or conditions like respiratory diseases such as asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, irritable bowel syndrome, gastrointestinal dysfunction, secretory diarrhoea, ischaemia, cerebral ischaemia, ischaemic hearth disease, angina pectoris, coronary hearth disease, traumatic brain injury, psychosis, anxiety, depression, dementia, memory and attention deficits, Alzheimer's disease, dysmenorrhea, narcolepsy, Reynaud's disease, intermittent claudication, Sjorgren's syndrome, migraine, arrhythmia, hypertension, absence seizures, myotonic muscle dystrophia, xerostomi, diabetes type II, hyperinsulinemia, premature labour, baldness, cancer and immune suppression.

Conditions which may benefit from this treatment include auto-immune related diseases, disorders or conditions, e.g. Addison's disease, alopecia areata, Ankylosing spondylitis, haemolytic anemia (anemia haemolytica), pernicious anemia (anemia perniciosa), aphthae, aphthous stomatitis, arthritis, arteriosclerotic disorders, osteoarthritis, rheumatoid arthritis, aspermiogenese, asthma bronchiale, auto-immune asthma, auto-immune hemolysis, Bechet's disease, Boeck's disease, inflammatory bowel disease, Burkitt's lymphoma, Bullous pemphigoid, Chron's disease, chorioiditis, colitis ulcerosa, Coeliac disease, cryoglobulinemia, Chronic inflammatory demyelinating polyneuropathy (CIDP), Cicatricial pemphigoid (also known as mucous membrane pemphigoid or benign pemphigoid), Churg-Strauss syndrome (also known as allergic granulomatosis), CREST syndrome (an acronym for calcinosis, Raynaud's phenomenon, esophageal dysfunction, sclerodactyly and telangiectasia), dermatitis herpetiformis, dermatomyositis, insulin-dependent type I diabetes, juvenile diabetes, idiopathic diabetes insipidus, insulin-dependent diabetes mellisis, auto-immune demyelinating diseases, Dupuytren's contracture, encephalomyelitis, encephalomyelitis allergica, endophthalmia phacoanaphylactica, enteritis allergica, auto-immune enteropathy syndrome, erythema nodosum leprosum, idiopathic facial paralysis, chronic fatigue syndrome, febris rheumatica, glomerulo nephritis, Goodpasture's syndrome, Graves' disease, Hamman-Rich's disease, Hashimoto's disease, Hashimoto's thyroiditis, sudden hearing loss, sensoneural hearing loss, hepatitis chronica, Hodgkin's disease, haemoglobinuria paroxysmatica, hypogonadism, ileitis regionalis, iritis, hyper-keratose, leucopenia, leucemia, lupus erythematosus disseminatus, systemic lupus erythematosus, cutaneous lupus erythematosus, lymphogranuloma malignum, mononucleosis infectiosa, myasthenia gravis, traverse myelitis, primary idiopathic myxedema, nephrosis, ophthalmia symphatica, orchitis granulomatosa, pancreatitis, pemphigus, pemphigus vulgaris, polyarteritis nodosa, polyarthritis chronica primaria, polymyositis, polyradiculitis acuta, psoreasis, purpura, pyoderma gangrenosum, Quervain's thyreolditis, Reiter's syndrome, restinosis, sarcoidosis, ataxic sclerosis, progressive systemic sclerosis, scieritis, sclerodermia, multiple sclerosis, sclerosis disseminata, acquired spenic atrophy, infertility due to antispermatozoan antobodies, thrombocytopenia, idiopathic thrombocytopenia purpura, thymoma, acute anterior uveitis, vitiligo, AIDS, HIV, SCID and Epstein Barr virus associated diseases such as Sjorgren's syndrome, virus (AIDS or EBV) associated B cell lymphoma, parasitic diseases such as Lesihmania, and immunosuppressed disease states such as viral infections following allograft transplantations, graft vs. Host syndrome, transplant rejection, or AIDS, cancers, chronic active hepatitis diabetes, toxic chock syndrome, food poisoning, and transplant rejection.

The compounds of the invention are also considered particularly useful for reducing or inhibiting undesired immune-regulatory actions. In a preferred embodiment, therefore, the compounds of the may be used in the treatment or alleviation of a diseases, disorders or condition related to immune dysfunction, or in order to obtain immune suppression in an individual in need therefore.

In a more preferred embodiment, the invention relates to the use of an IK_{Ca} inhibitory compound of the invention in a combination therapy with known immune-suppressants for the treatment or alleviation of a diseases, disorders or condition related to immune dysfunction, or for obtaining immune suppression. Preferred immune-suppressants to combine with the compounds of the invention include Amphotericin, Busulphan, Co-trimoxazole, Chlorambucil, colony stimulating factors, corticosteroids, Cyclophosphamide, Fluconazole, folinic acid, Ganciclovir, antilymphocyte immunoglobulins, normal immunoglobulins, Methotrexate, Methylprednisolone, Octreotide, Oxpentifylline, Tacrolimus (FK506), Thalidomide, Zolimomab aritox, and the calcineurin inhibitors (protein phosphatase 2B inhibitors), in particular Cyclosporin.

Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical— compound of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depend on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Methods of Therapy

In another aspect the invention provides a method for the treatment, prevention or alleviation of a disease or a disorder or a condition of a living animal body, including a human, which disease, disorder or condition is responsive to inhibition of SK_{Ca} and/or IK_{Ca} channels, and which method comprises administering to such a living animal body, including a human, in need thereof an effective amount of a chemical compound of the invention.

The preferred indications contemplated according to the invention are those stated above.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Preparatory Example

### 2,2-Bis-(4-fluoro-phenyl)-succinamide (Compound 1)

1,2-di-Cyano-2,2-di-p-fluorophenyl-ethane (930 mg) was heated in conc. sulphuric acid (5 ml) at 50° C. for 1 hour. The reaction mixture was poured onto ice and neutralized with dil. aq. potassoium hydroxide. The mixture was extracted with diethyl ether (3×25 ml) and the etheral extracts were dried over Na₂SO₄, filtered and concentrated in vacuo. The solid residue was flash-chromatographed and the low running product (R_{f}=0.1/hexane:EtOAc:MeOH, 10:3:1.5) was recrystallised from EtOH to afford the product as white plates (110 mg). M.p. 192-193° C.

### 3-Cyano-3,3-bis-(4-fluoro-phenyl)-propionic acid (Compound 2)

After flash-chromatography, the higher R_{f} product from the above reaction (R_{f}=0.5/hexane:EtOAc:MeOH, 10:3:1.5) was recrystallised from EtOH to afford the product as white plates (90 mg). M.p.=165-166° C.

### p-Toluensulfonic acid 2-cyano-2,2-bis-(4-fluoro-phenyl)-ethyl ester (Compound 3)

To a magnetically stirred solution of 2-cyano-2,2-di-p-fluorophenyl-ethanol (518 mg) in dry methylene chloride (5 ml) were added sequentially, under argon and at room temp. DMAP (145 mg), tosyl chloride (458 mg) and triethylamine (0.2 ml). The reaction mixture was stirred at room temp for 2 hours and then diluted with diethyl ether (20 ml). Filtration of the precipitate, sequential washing of the filtrate solution with 10% aq. CuSO₄, 10% aq. NaHCO₃ and brine, drying (Na₂SO₄), filtration and concentration in vacuo provided a product which was recrystallised from EtOH to afford the tosylate (532 mg; 64%). M.p.=120-121° C.

### Ethyl 4-cyano-4,4-bis-(4-fluoro-phenyl) butyrate (Compound 4)

A solution of di-p-fluorophenyl ethane nitrile (1 g) in dry THF (25 ml) at room temperature under argon was treated with Triton-B (0.09 equiv.) and after 10 min ethyl acrylate (0.47 ml) in dry THF (1 ml) was added dropwise. Stirring continued for 5 hours before acidification with dil. aq. HCl. Extraction with diethyl ether, drying (Na₂SO₄), filtration and concentration gave a residue which was flash-chromatographed (eluent: EtOAc:hexane, 1:6) to afford the product (450 mg; 32%) as a colourless oil.

### Methyl 4-cyano-4,4-bis-(pyridin-2-yl) butyrate (Compound 5)

A solution of di-pyridin-2-yl ethane nitrile (390 mg) in tert.-butanol (2 ml) at room temperature under argon was treated with Triton-B (0.09 equiv.) and after 10 minutes methyl acrylate (0.22 ml) was added dropwise. Stirring continued for 30 minutes at 60° C. and the solvent was removed in vacuo. The resulting oil was taken up in chloroform, washed with dil. aq. HCl, aq. sodium carbonate and water. Drying (MgSO₄), filtration and concentration in vacuo gave a residue which was flash-chromatographed (eluent: EtOAc:hexane, 1:2) to afford the product (170 mg; 30%) as a white solid. M.p. 59-60° C.

### 2-[(2-Fluoro-phenyl)-bis-(4-fluoro-phenyl)-methanesulfanyl]-N-hydroxy acetamidine (Compound 6)

Metallic sodium (62 mg) was dissolved in dry methanol under an Argon atmosphere. When the metal had reacted, (2-Fluoro-phenyl-bis-4-fluoro-phenyl)-methanesulfanyl-acetonitrile (500 mg; 1.35 mmol) and hydroxylamine hydrochloride (188 mg; 2.71 mmol) were added and the mixture was stirred at 50° C. for 18 hours. Filtration and concentration afforded a residue which was taken up in chloroform, filtered again and concentrated. Flash chromatography using, sequentially, petroleum ether:ethyl acetate in the proportions 8:2, 7:3 and 6:4 gave a colourless gum (192 mg; 35%).

### Methyl 4-cyano-2-methyl-4,4-bis-(pyridin-2-yl) butyrate (Compound 7)

A procedure similar to that for the preparation of Compound 5, using methyl methacrylate and 5 hours of reflux for the reaction, provided, after flash-chromatography (eluent: EtOAc:hexane, 1:1), the product (190 mg; 32%) as a white solid. M.p. 44-45° C.

### 2-(4Fluoro-phenyl)-2-(4-nitro-3-trifluoromethyl-phenyl)-3-pyridin-2-yl-propionitrile (Compound 8)

2-(4-Fluorophenyl)-3-pyridin-2-ylpropionitrile (990 mg) dissolved in DMF (15 ml) was treated with 60% NaH (180 mg) at 0° C. After 2 hours 4-fluoro-6-trifluoromethyl nitrobenzene (0.7 ml) was added. The reaction was heated at 55-60° C. overnight, cooled to room temperature, treated with more 60% NaH (160 mg) and 4-fluoro-6-trifluoromethyl nitrobenzene (0.6 ml) and heated again to 60° C. for 12 hours and to 80° C. for 24 hours. The reaction was quenched with ammonium chloride solution, acidified with diluted HCl and extracted with EtOAc (3×). Drying (MgSO₄), filtration and concentration afforded a residue (2.4 g), which was chromatographed (eluent: EtOAc/Petrol, 1:3; then EtOAc/Petrol, 1:2; then EtOAc/Petrol, 1:1; then pure EtOAc) to give a product (1.1 g) which was taken up in Et₂O and precipitated with petrol, affording the desired 2-(4-Fluoro-phenyl)-2-(4-nitro-3-trifluoromethyl-phenyl)-3-pyridin-2-yl-propionitrile (960 mg, 52%). M.p. 118-119° C.

### Example 2

Biological Activity

Block of IK Channels

In this example the ability of the compounds of the invention to inhibit human intermediate-conductance Ca²⁺-activated K⁺ channels (hIK channels) is examined.

hIK channels have been cloned from human placenta and stably expressed in HEK293 cells. The ionic current through the channels is recorded in the whole-cell mode of the patch-clamp technique.

A Kᵢ value is calculated from the kinetics of the block.

The inhibition of the current is assumed to occur by a simple drug (D)−receptor (R) interaction after the following scheme:

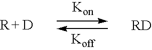

This is a simple bimolecular reaction, which integrated under non-equalibrium conditions are described by the equation:
Iₜ=I₀*(1−(C/C+(k_{off}/kₒₙ)))*(1−exp(−(C* kₒₙ+k_{off})*t)))
wherein,
Iₜ=current at time t in nA
k_{off}=off-rate in s⁻¹
I₀=basal current in nA
kₒₙ=on-rate in M⁻¹s⁻¹
C=drug concentration in μM

By using the equation above, a fit to the time-course of the decrease in current yields the values k_{off} and kₒₙ. Kᵢ equals the ratio k_{off}/kₒₙ, and Kᵢ is the test value to be reported. At t=∞ (equalibrium) the equation simplifies to the Michaelis-Menten equation with Kᵢ=IC₅₀.

The results of this experiment are presented in Table 1, below, and show activity in the low micromolar range.

TABLE 1
Block of IK Channels

| Compound No. | Kᵢ (μM) |
|---|---|
| 2 | 2.2 |
| 4 | 1.4 |
| 6 | 0.53 |
| 8 | 0.065 |

1. A diaryl methyl derivative of Formula I 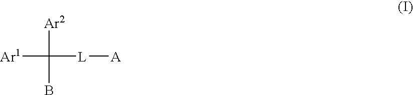
or a pharmaceutically-acceptable addition salt thereof, wherein,
Ar¹ and Ar², independently of one another, represent an aromatic carbocyclic or heterocyclic monocyclic group, which aromatic carbocyclic or heterocyclic monocyclic group is optionally substituted one or more times with substituents selected from the group consisting of alkyl, alkoxy, halo, haloalkyl, haloalkoxy, cyano and nitro;
L is absent (i.e. represents a single bond) or represents a linker selected from the group consisting of —CH₂—, —CH₂CH₂—, —CH(CH₃)—, —CH₂CH₂CH₂—, —CH₂CH(CH₃)—, —S—, —S—CH₂—, —S—CH₂CH₂—, —S—CH(CH₃)—, —S—CH₂CH₂CH₂—, —S—CH₂CH(CH₃)—, —NH—, —NH—CH₂—, —NH—CH₂CH₂—, —NH—CH(CH₃)—, —NH—CH₂CH₂CH₂— and —NH—CH₂CH(CH₃)—; and
A and B, independently of one another, represent
—CN; —COOR′, —CONR′R″, —C(═NOR′)R″ or —C(═NOR′)NR″R′″, wherein R′, R″ and R′″, independently of one another, represent hydrogen or alkyl;
pyridinyl, phenyl, —SO₂-phenyl or —O—SO₂-phenyl, which phenyl group may optionally be substituted one or more times with substituents selected from the group consisting of alkyl, alkoxy, halo, haloalkyl, haloalkoxy, cyano and nitro; or
A represents —CN; —COOR′, —CONR′R″, —C(═NOR′)R″ or —C(═NOR′)NR″R′″, wherein R′, R″ and R′″, independently of one another, represent hydrogen or alkyl; pyridinyl, —SO₂-phenyl or —O—SO₂-phenyl, which phenyl group may optionally be substituted with alkyl, alkoxy, halo, haloalkyl, haloalkoxy, cyano and nitro; and
B represents —CN, —COOR′ or —CONR′R″, wherein R′ and R″, independently of one another, represent hydrogen or alkyl; a phenyl group, which phenyl group is optionally substituted one or two times with alkyl, alkoxy, halo, haloalkyl, haloalkoxy, cyano and/or nitro; or a pyridinyl group, which pyridinyl group is optionally substituted one or two times with alkyl, alkoxy, halo, haloalkyl, haloalkoxy, cyano and/or nitro.

2. The compound of claim 1, wherein
L represents a linker selected from the group consisting of —CH₂—, —CH₂CH₂—, —CH₂CH₂CH₂—, —CH₂CH(CH₃)—, —S—, —S—CH₂— and —S—CH(CH₃)—.

3. The compound of claim 1, wherein
A and B, independently of one another, represent
—CN; —COOR′, —CONR′R″, —C(═NOR′)R″ or —C(═NOR′)NR″R′″, wherein R′, R″ and R′″, independently of one another, represent hydrogen or alkyl;
pyridinyl, phenyl, —SO₂-phenyl or —O—SO₂-phenyl, which phenyl group may optionally be substituted one or more times with substituents selected from the group consisting of alkyl, alkoxy, halo, haloalkyl, haloalkoxy, cyano and nitro.

4. The compound of claim 1, wherein
A represents —CN; —COOR′, —CONR′R″, —C(═NOR′)R″ or —C(═NOR′)NR″R′″, wherein R′, R″ and R′″, independently of one another, represent hydrogen or alkyl; pyridinyl, —SO₂-phenyl or —O—SO₂-phenyl, which phenyl group may optionally be substituted with alkyl, alkoxy, halo, haloalkyl, haloalkoxy, cyano and nitro; and
B represents —CN, —COOR′ or —CONR′R″, wherein R′ and R″, independently of one another, represent hydrogen or alkyl; a phenyl group, which phenyl group is optionally substituted one or two times with alkyl, alkoxy, halo, haloalkyl, haloalkoxy, cyano and/or nitro; or a pyridinyl group, which pyridinyl group is optionally substituted one or two times with alkyl, alkoxy, halo, haloalkyl, haloalkoxy, cyano and/or nitro.

5. The compound of claim 4, wherein
A represents —COOH, —COOCH₃, —COOCH₂CH₃, —CONH₂, —C(═NOH)NH₂, pyridinyl or —O—SO₂-phenyl, which phenyl group is substituted with alkyl or halo, haloalkyl, cyano or nitro; and
B represents —CONH₂, —CN, or a phenyl group substituted with fluoro.

6. The compound of claim 5, wherein
A represents —COOH, —COOCH₃, —COOCH₂CH₃, —CONH₂, —C(═NOH)NH₂; and
B represents —CONH₂, —CN.

7. The compound of claim 5, wherein
A represents pyridin-2-yl, pyridin-3-yl, pyridin-4-yl or —O—SO₂-phenyl, which phenyl group is substituted with methyl or ethyl; and
B represents —CN.

8. The compound of claim 1, wherein
Ar¹ and Ar², independently of one another, represent
a phenyl group, which phenyl group is substituted one or two times with alkyl, alkoxy, halo, haloalkyl, cyano and/or nitro; or
a pyridinyl group, which pyridinyl group is optionally substituted one or two times with alkyl, alkoxy, halo, haloalkyl, cyano and/or nitro.

9. The compound of claim 8, wherein
both of Ar¹ and Ar² represent a phenyl group, which phenyl groups, independently of one another, are substituted one or two times with halo, haloalkyl, cyano and/or nitro; or
both of Ar¹ and Ar² represent a pyridinyl group, which pyridinyl groups, independently of one another, are optionally substituted one or two times with halo, haloalkyl, cyano and/or nitro.

10. The compound of claim 9, wherein
both of Ar¹ and Ar² represent a halo-substituted phenyl group.

11. The compound of claim 8, wherein
L represents —CH₂—, —CH₂CH₂—, —CH₂CH₂CH₂—, —CH₂CH(CH₃)—, —S—, —S—CH₂— or —S— CH(CH₃)—.

12. The compound of claim 8, wherein
A represents —COOH, —COOCH₃, —COOCH₂CH₃, —CONH₂, —C(═NOH)NH₂; and
B represents —CONH₂, —CN.

13. The compound of claim 10, which is
2,2-Bis-(4-fluoro-phenyl)-succinamide;
3-Cyano-3,3-bis-(4-fluoro-phenyl)-propionic acid;
p-Toluensulfonic acid 2-cyano-2,2-bis-(4-fluoro-phenyl)-ethyl ester;
Ethyl 4-cyano-4,4-bis-(4-fluoro-phenyl) butyrate; or
2-[(2-Fluoro-phenyl)-bis-(4-fluoro-phenyl)-methanesulfanyl]-N-hydroxy acetamidine;
or a pharmaceutically-acceptable addition salt thereof.

14. The compound of claim 9, wherein both of Ar¹ and Ar² represent a phenyl group, which phenyl groups, independently of one another, are substituted one or two times with halo, haloalkyl, cyano and/or nitro.

15. The compound of claim 14, wherein
L represents —CH₂—, —CH₂CH₂—, —CH₂CH₂CH₂—, —CH₂CH(CH₃)—, —S—, —S—CH₂— or —S— CH(CH₃)—.

16. The compound of claim 14, wherein
A represents pyridinyl, in particular pyridin-2-yl, pyridin-3-yl or pyridin-4-yl; and
B represents —CONH₂, —CN.

17. The compound of claim 14, which is
2-(4-Fluoro-phenyl)-2-(4-nitro-3-trifluoromethyl-phenyl)-3-pyridin-2-yl-propionitrile;
or a pharmaceutically-acceptable addition salt thereof.

18. The compound of claim 9, wherein both of Ar¹ and Ar² represent a pyridinyl, in particular a pyridin-2-yl, a pyridin-3-yl or a pyridin-4-yl group.

19. The compound of claim 18, wherein
L represents —CH₂—, —CH₂CH₂—, —CH₂CH₂CH₂—, —CH₂CH(CH₃)—, —S—, —S—CH₂— or —S— CH(CH₃)—.

20. The compound of claim 18, wherein
A represents —COOH, —COOCH₃, —COOCH₂CH₃, —CONH₂, —C(═NOH)NH₂; and
B represents —CONH₂, —CN.

21. The compound of claim 18, which is
Methyl 4-cyano-4,4-bis-(pyridin-2-yl) butyrate; or
Methyl 4-cyano-2-methyl-4,4-bis-(pyridin-2-yl) butyrate;
or a pharmaceutically-acceptable addition salt thereof.

22. A pharmaceutical composition comprising a therapeutically effective amount of a compound of claim 1, or a pharmaceutically-acceptable addition salt thereof.

23. A method for of treatment, prevention or alleviation of a disease or a disorder or a condition responsive to modulation of SK_{Ca} and/or IK_{Ca} channels, which method comprises the step of administering to such a living animal body in need thereof a therapeutically effective amount of a compound of claim 1.

24. The method according to claim 23, which disease, disorder or condition relates to reduction or inhibition of undesired immune-regulatory actions, including graft vs. host syndrome, transplant rejection, or transplant rejection.

25. The method according to claim 23, for the manufacture of a pharmaceutical composition, which further comprises a pharmaceutically effective amount of a conventional immune suppressing agent.

26. The method according to claim 25, wherein the conventional immune-suppressing agent is Amphotericin, Busulphan, Co-trimoxazole, Chlorambucil, colony stimulating factors, corticosteroids, Cyclophosphamide, Fluconazole, folinic acid, Ganciclovir, antilymphocyte immunoglobulins, normal immunoglobulins, Methotrexate, Methylprednisolone, Octreotide, Oxpentifylline, Tacrolimus (FK506), Thalidomide, Zolimomabaritox, or the calcineurin inhibitors (protein phosphatase 2B inhibitors), in particular Cyclosporin.

27. (canceled)
